(19) United States
(12) Patent Application Publication
(10) Pub. No.: US 2004/0147799 A1
(43) Pub. Date: Jul. 29, 2004

(54) Catheter unit for radiation therapy

(75) Inventor: Makoto Inaba, Kunitachi-shi (JP)

Correspondence Address: OSTROLENK FABER GERB & SOFFEN, 1180 AVENUE OF THE AMERICAS, NEW YORK, NY, 100368403

(73) Assignee: Olympus Corporation

(21) Appl. No.: 10/713,398

(22) Filed: Nov. 14, 2003

(30) Foreign Application Priority Data
Nov. 15, 2002 (JP) 2002-332175
Oct. 27, 2003 (JP) 2003-366308

(51) Int. Cl. A61N 5/00; A61B 1/00

(52) U.S. Cl. 600/3; 600/145; 600/104

(57) ABSTRACT

A catheter unit comprising an elbowed catheter having an insert section capable of being inserted into the body cavity and an elbow section and a winged catheter capable of being attached to the elbowed catheter so as to cover the insert section of the elbowed catheter. The two catheters are introduced into the body cavity by utilizing the bending function and fixed in the body cavity by means of a wing portion attached to the winged catheter.

### CROSS-REFERENCE TO RELATED APPLICATIONS

This application is based upon and claims the benefit of priority from the prior Japanese Patent Applications No. 2002-332175, filed Nov. 15, 2002; and No. 2003-366308, filed Oct. 27, 2003, the entire contents of both of which are incorporated herein by reference.

### BACKGROUND OF THE INVENTION

1. Field of the Invention

The present invention relates to a catheter unit for guiding a tube, which leads a radiation source into the body cavity in intraluminal radiation therapy, into the body cavity.

2. Description of the Related Art

In intraluminal radiation therapy that employs a radiation source, a catheter is used to introduce a radiation source tube into a cavity. The catheter has wings on its distal end, whereby it is fixed in a predetermined position in the cavity. Use of the catheter conventionally requires the following steps of operation, as is disclosed in “Lung Cancer”, Japanese Journal of Clinical Radiology, Vol. 41, No. 13, 1996.

1. An endoscope is inserted into a bronchus through one nostril.

2. A radiation source tube is inserted into a channel of the endoscope.

3. The endoscope is removed with the radiation source tube left in the bronchus.

4. After the endoscope is inserted into the bronchus through another nostril or the mouth, it is used for observation from the backside as the winged catheter is inserted into the bronchus with the radiation source tube used as a guide.

5. The catheter is observed through the endoscope as its wings are spread in a desired position so that the catheter is fixed in the desired position.

### BRIEF SUMMARY OF THE INVENTION

According to an aspect of the invention, there is provided a catheter unit which guides a tube, which leads a radiation source used in intraluminal radiation therapy into a body cavity, into the body cavity, comprising: a first catheter which has an insert section capable of being inserted into the body cavity, an elbow section attached to the distal end portion of the insert section and bendable, and a bending control section which bends the elbow section, and guides the tube; and a second catheter attached to the first catheter in a manner such that the insert section penetrates the second catheter and the elbow section penetrates, the second catheter having at least one wing portion, attached to a part of the second catheter and capable of spreading to be anchored in the body cavity and of being opened and closed, and a control section capable of opening and closing the wing portion.

According to another aspect of the invention, there is provided a method of guiding a tube, which leads a radiation source into a living body cavity, into the body cavity through a conduit of a catheter so that the radiation source administers radiation therapy to the interior of the body cavity, comprising: a step of setting the tube in the conduit of the catheter; a step of inserting the catheter, having the tube set in the conduit thereof, into the living body cavity; a step of inserting an endoscope into the body cavity and observing the position of the catheter and the state of insertion as the catheter is inserted into the body cavity; and a step of opening the wing portion of the catheter which reduces the eccentricity of the tube in the cavity and settles the position of detention of the catheter in the cavity.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a perspective view showing an outline of an assembled catheter unit according to an embodiment of the invention;

FIG. 2 is a perspective view showing an outline of an elbowed catheter of the catheter unit according to an embodiment of the invention;

FIG. 3 is a cutaway perspective view showing an outline of a winged catheter of the catheter unit according to the embodiment of the invention;

FIG. 4 is a side view showing a profile of a region near the distal end portion of the winged catheter with its wing portion spread according to the embodiment of the invention;

FIG. 5 is a side view showing a profile of the region near the distal end portion of the winged catheter with its wing portion retracted according to the embodiment of the invention;

FIG. 6 is a view illustrating a system for administrating radiation therapy to the interior of the body cavity with use of the catheter unit; and

FIG. 7 is a view illustrating steps of procedure for the radiation therapy using the catheter unit.

### DETAILED DESCRIPTION OF THE INVENTION

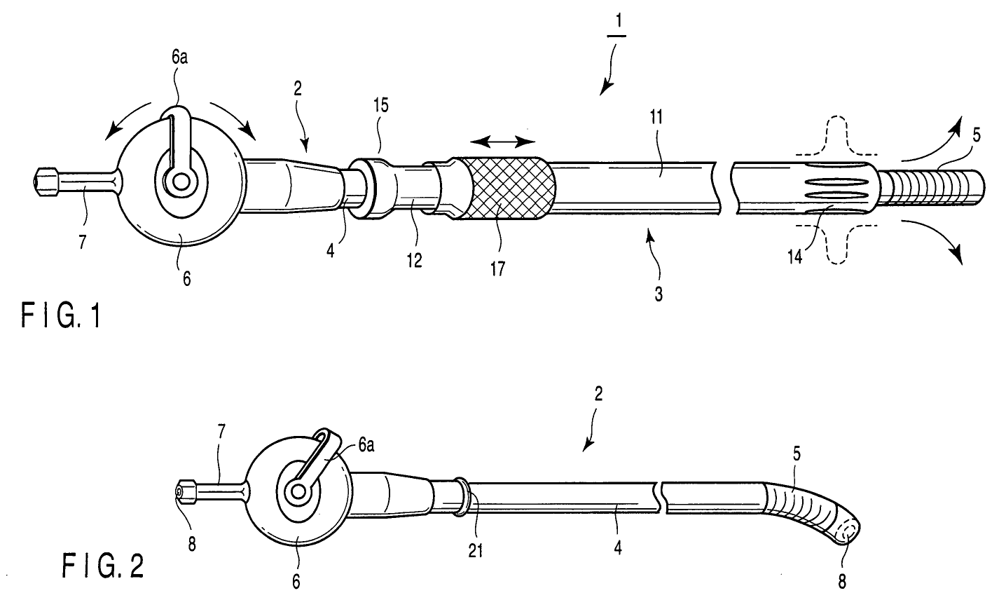

A catheter unit according to an embodiment of the present invention, a radiotherapy system using the unit, and an example of use of the system will now be described with reference to FIGS. 1 to 7.

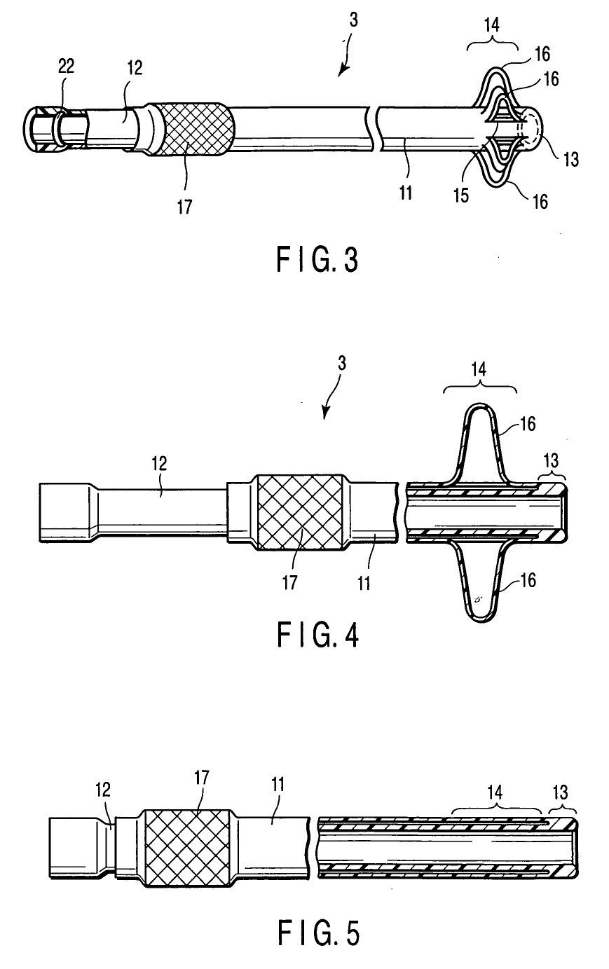

A catheter unit 1 according to the present embodiment combines an elbowed catheter 2 shown in FIG. 2 and a winged catheter 3 shown in FIG. 3 in the manner shown in FIG. 1.

As shown in FIG. 2, the elbowed catheter 2 comprises an insert section 4, which is formed of a flexible sheath. An elbow section 5 is attached to the distal end portion of the insert section 4. A bending control section 6 is coupled to the proximal end of the insert section 4. A hold section 7 is provided integrally on the proximal end of the bending control section 6. The elbowed catheter 2 is penetrated by a conduit 8 that extends from the proximal end of the hold section 7 to the distal end of the elbow section 5. A radiation source tube 9 (mentioned later) or any other manipulator, such as a guide wire (not shown), can be passed through the conduit 8.

As shown in FIGS. 3 to 5, the winged catheter 3 is provided with an outer sheath 11 and an inner sheath 12, both of which are formed of resin. The inner sheath 12 is fitted in the outer sheath 11. The catheter 3 has a double-sheath structure that combines the outer and inner sheaths 11 and 12. The outer and inner sheaths 11 and 12 can move back and forth in sliding contact with each other. As shown in FIG. 4, the respective distal ends of the outer and inner sheaths 11 and 12 are coupled integrally by means of a coupling portion 13. Since these distal ends are directly coupled in this manner, the length of a rigid part of the distal end portion of the winged catheter 3 can be shortened.

As shown in FIGS. 3 and 4, a retaining portion 14 for fixing the winged catheter 3 in a predetermined position in a cavity is provided on that part of the distal end portion of the outer sheath 11 which is situated on the hand side of the coupling portion 13. The retaining portion 14 includes a plurality of wings 16 that are integral with the sheath member that forms the outer sheath 11. The wings 16 are defined by forming a plurality of longitudinal slits 15 in the sheath member itself. If the wings 16 are formed on the sheath member, they cover the whole region of the retaining portion 14 except some parts of the sheath member, as shown in FIG. 3. Alternatively, however, the whole region of the sheath member may be divided in a plurality of parts (not shown) that form the wings 16, individually.

As shown in FIGS. 3 to 5, a sliding control section 17, which can be grasped by a hand, is coupled to the proximal end portion of the outer sheath 11. If the outer sheath 11 is advanced with respect to the inner sheath 12 with the sliding control section 17 in a hand, the region of the retaining portion 14 contracts, and all the wings 16 spread outward and open, as indicated by full lines in FIGS. 3 and 4. If the sliding control section 17 is retreated, on the other hand, the region of the retaining portion 14 having the wings 16 extend straight so that all the wings 16 close, as indicated by full lines in FIGS. 1 and 5. Then, the region of the retaining portion 14 shrivels, and the wings 16 are kept retracted. By manipulating the sliding control section 17, the wings 16 that are situated near the distal end from the winged catheter 3 can be opened or closed to expand or contract the retaining portion 14. Usually, each wing 16 is a relatively flexible structure having elasticity.

Removable anchor means is located near both the respective proximal ends of the insert section of the elbowed catheter 2 and the winged catheter 3. This anchor means fixes the two catheters 2 and 3 in a predetermined position where they are combined. The anchor means is a click mechanism that combines an annular protuberance 21 and an annular groove 22 in which the protuberance 21 can be fitted. The protuberance 21 is formed on the outer periphery of the elbowed catheter 2 near the proximal end of the insert section, as shown in FIG. 2. As shown in FIG. 3, on the other hand, the groove 22 is formed in the inner surface of the winged catheter 3 near its proximal end. The catheters 2 and 3 can be removably attached to each other, since they are caused to engage by utilizing the elasticity of the protuberance 21 and the groove 22. Alternatively, the anchor means may be formed of a combination of tapered structures that are joined together by caulking or screwing. Further, the anchor means may be located near the respective distal ends of the insert section 4 of the elbowed catheter 2 and the winged catheter 3. Furthermore, the anchor means may be located in any other region where the catheters 2 and 3 engage each other.

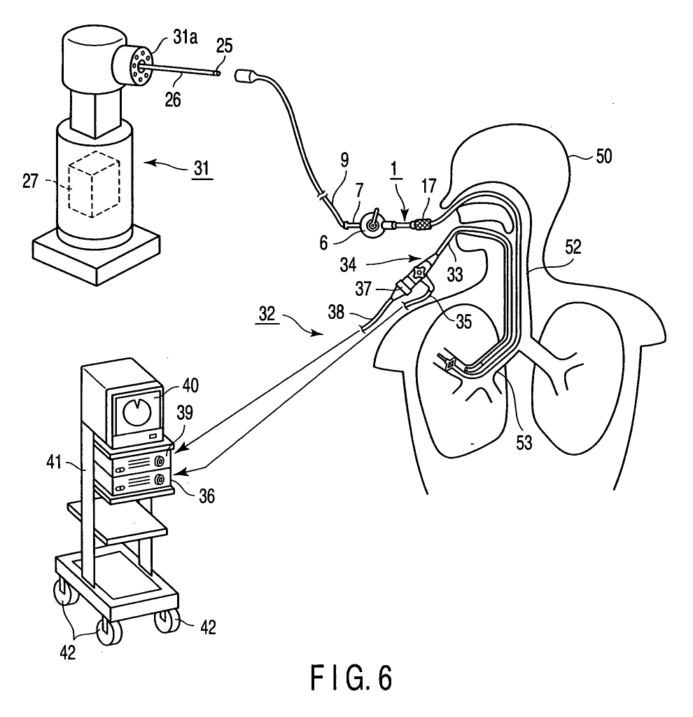

A system for treating the interior of the body cavity with radiation using the catheter unit 1 will now be described with reference to FIG. 6.

This system comprises an intraluminal radiator 31 and an endoscope system 32, besides the catheter unit 1. The radiation source tube 9 is connected to the intraluminal radiator 31. The tube 9 is connected to a connecting port 31a of the radiator 31. It guides a radiation source 25 supplied from the radiator 31 and leads it into the body cavity to which its distal end is confined. As shown in FIG. 6, the radiation source 25 is attached to a distal end of a thin wire 26 that can be passed through the radiation source tube 9. The intraluminal radiator 31 can hold therein the wire 26, which is fitted with the radiation source 25, and incorporates a delivery device 27 that can deliver the wire 26 through the radiation source tube 9 that is connected to the connecting port 31a.

Iridium is used for the radiation source 25. Alternatively, a small radiation source, such as cobalt, may be used for the purpose, depending on the case of the radiation therapy.

As shown in FIG. 6, the endoscope system 32 comprises an endoscope 34 having an elongate insert section 33 and a light source unit 36 to be connected with a light guide cord 35 of the endoscope 34. The system 32 further comprises a camera unit 39 to be connected with a signal cord 38 and a monitor 40 for displaying an image that is picked up by means of the endoscope 34. The signal cord 38 is connected to a camera head 37 that is attached to the endoscope 34. The light source unit 36, camera unit 39, monitor 40, and other peripheral devices are set on a rack 41. Having casters 42, the rack 41 can move on the floor.

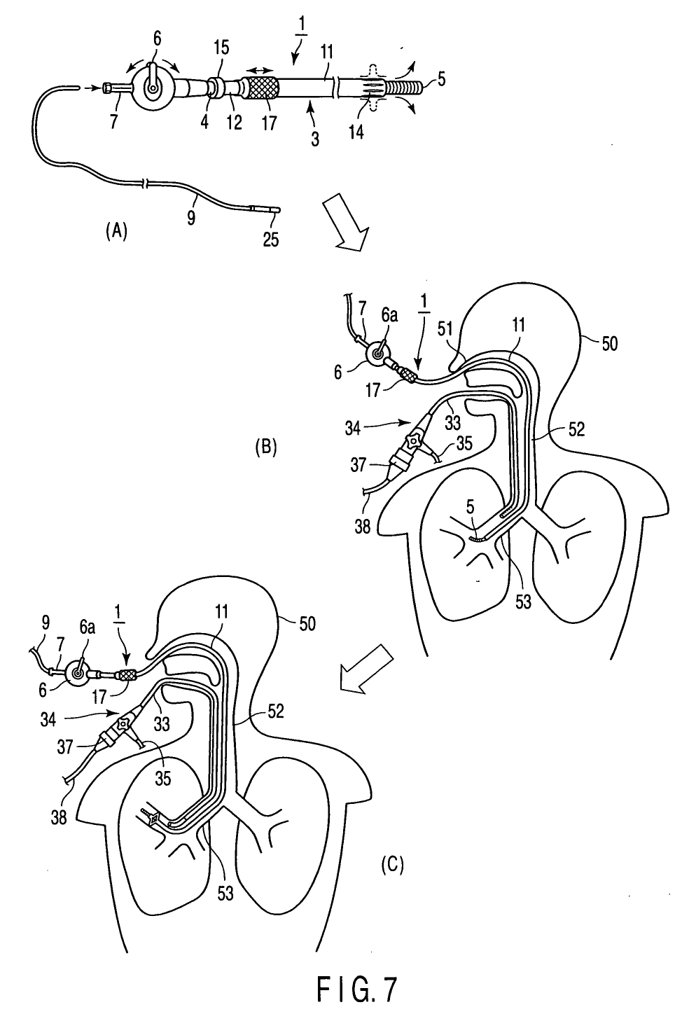

The following is a description of an example of use of the intraluminal radiotherapy system. First, the winged catheter 3 is fitted into the elbowed catheter 2 to assemble the catheter unit 1, as shown by (A) in FIG. 7. When the catheter unit 1 is assembled in this manner, the winged catheter 3 allows the elbow section 5 of the elbowed catheter 2 to be exposed, and covers the insert section 4 of the elbowed catheter. Further, the protuberance 21 and the groove 22 of the anchor means engage each other, so that the two catheters 2 and 3 are fixedly held in the predetermined position.

After the catheter unit 1 is assembled in this manner, the radiation source tube 9 for intraluminal small-radiation-source therapy is inserted into the conduit 8 of the elbowed catheter 2, as shown by (A) in FIG. 7. The insert section of the catheter unit 1, thus fitted with the radiation source tube 9, is inserted into a bronchus 53 through one nostril 51 and a trachea 52 of a patient 50, as shown by (B) in FIG. 7.

As shown by (B) in FIG. 7, moreover, the insert section 33 of the endoscope 34 is inserted into the bronchus 53 through the other nostril, mouth, or tracheostomy (opening formed in the cervical region in a tracheotomic manner). As shown by (B) and (C) in FIG. 7, the state of the insert section of the catheter unit 1 is observed from the backside through the endoscope 34 that is inserted nasally, orally, or through a tracheotomy tube into the living body cavity as the distal end of the insert section of the winged catheter 3 is guided deep into the bronchus 53. In doing this, the whole catheter unit 1 is impelled with the distal end of the elbowed catheter 2 directed in a desired direction by utilizing the function of the catheter 2 to bend the elbow section 5. Thus, the distal end portion of the winged catheter 3 is inserted deep into the bronchial lumen.

If it is confirmed by observing the catheter unit 1 by means of the endoscope 34 that the unit 1 is in a desired position, as shown by (C) in FIG. 7, only the sliding control section 17 of winged catheter 3 is slid forward. When this is done, the wings 16 of the winged catheter 3 spread to be anchored to the lumen wall of the bronchus 53, whereupon the catheter 3 is fixed in the bronchus 53. Further, the hold section 7 of the elbowed catheter 2 is grasped by means of a holding device, such as a holder, and is held in the predetermined position in which the catheter unit 1 is set. Thereafter, the endoscope 34 is drawn out.

Thus, the delivery device 27 of the intraluminal radiator 31 is driven with only the catheter unit 1 held in the body cavity, and the radiation source 25, along with the wire 26, is introduced from the radiator 31 into a region near the distal end of the catheter unit 1 through the radiation source tube 9. The intraluminal radiation therapy is administered in this state.

According to the present embodiment, as described above, the elbowed catheter 2 that has the bending function and the winged catheter 3 are combined together to form the catheter unit 1. Therefore, the distal end of the catheter unit 1 that leads the radiation source tube 9 and the like can be easily guided to a treatment position in the cavity. Further, objects, such as the radiation source tube 9 and the endoscope to be inserted into the cavity, can be easily or smoothly inserted into and removed from the cavity.

Thus, burdens on the operator and the patient can be lightened considerably. Since the observation through the endoscope is effective, moreover, fluoroscopic operation can be avoided or lessened, so that exposure of the patient to X-rays can be avoided or reduced. Further, the wing portion of the winged catheter 3 is more susceptible to deterioration than any other catheters. Since the winged catheter 3 can be replaced singly, however, the elbowed catheter 2 can be utilized as it is, so that the catheter unit 1 can be reused to ensure good economy.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A catheter unit which guides a tube, which leads a radiation source used in intraluminal radiation therapy into a body cavity, into the body cavity, comprising:
a first catheter which has an insert section capable of being inserted into the body cavity, an elbow section attached to the distal end portion of the insert section and bendable, and a bending control section which bends the elbow section, and guides the tube; and
a second catheter attached to the first catheter, holding the insert section, said second catheter comprising at least one retaining portion which is to be opened and closed, and a control section which is configured to open and close the retaining portion to be anchored in the body cavity when opened by the control section.

2. A catheter unit according to claim 1, which further comprises an anchor section which removably anchors the first and second catheters to each other, with the second catheter fitted on the first catheter and the two catheters assembled in a predetermined position.

3. A catheter unit according to claim 1, which further comprises a hold section attached to the first catheter and designed to be held to support the catheter unit.

4. A catheter unit according to claim 1, wherein the second catheter has an outer sheath and an inner sheath coupled to each other on the distal end side with respect to the retaining portion, and the retaining portion opens and closes as the outer sheath is slid relative to the inner sheath in the axial direction of the catheter.

5. A catheter unit according to claim 4, wherein the outer sheath has a plurality of slits cut in the axial direction of the first catheter so that regions between the slits define the retaining portion.

6. A catheter unit which guides a tube, which leads a radiation source used in intraluminal radiation therapy into a body cavity, comprising:
a first catheter which has an insert section capable of being inserted into the body cavity, an elbow section attached to the distal end portion of the insert section and bendable, and a bending control section which bends the elbow section, and guides the tube; and
a second catheter attached to the first catheter, holding the insert section and maintaining the elbow section in extending state, said second catheter comprising at least one retaining portion which is to be opened and closed, and operation means for opening and closing the retaining portion, said retaining portion being anchored in the body cavity when opened by the operation means.

7. A catheter unit according to claim 6, which further comprises anchor means for removably anchoring the first and second catheters to each other, with the second catheter fitted on the first catheter and the two catheters assembled in a predetermined position.

8. A catheter unit according to claim 6, which further comprises holding means attached to the first catheter and designed to be held to support the catheter unit.

9. A catheter unit according to claim 6, wherein the second catheter has an outer sheath and an inner sheath coupled to each other on the distal end side with respect to the retaining portion, and has opening/closing means for opening and closing the retaining portion as the outer sheath is slid relative to the inner sheath in the axial direction of the catheter.

10. A catheter unit according to claim 9, wherein the outer sheath has a plurality of slits cut in the axial direction of the first catheter so that regions between the slits define the retaining portion.

11. A method of guiding a tube, which leads a radiation source into a living body cavity, into the body cavity through a conduit of a catheter so that the radiation source administers radiation therapy to the interior of the body cavity, comprising:
a step of setting a tube in the conduit of the catheter;
a step of inserting the catheter, having the tube set in the conduit thereof, into the living body cavity;
a step of inserting an endoscope into the body cavity and observing the position of the catheter and the state of insertion as the catheter is inserted into the body cavity; and
a step of opening a retaining portion of the catheter to reduce the eccentricity of the tube in the cavity and settle the position of detention of the catheter in the cavity.
